(19) United States
(12) Patent Application Publication
(10) Pub. No.: US 2024/0358882 A1
(43) Pub. Date: Oct. 31, 2024

(54) FIXING STRUCTURE OF ULTRAVIOLET LAMP TUBE OF AIR DISINFECTION MACHINE

(71) Applicant: SOTO Air Purification Technology (Langfang) Co., Ltd., Langfang (CN)

(72) Inventors: Zhonghao PIAO, Langfang (CN); Hui LI, Langfang (CN)

(21) Appl. No.: 18/770,551

(22) Filed: Jul. 11, 2024

Related U.S. Application Data
(63) Continuation of application No. PCT/CN2023/070165, filed on Jan. 3, 2023.

(30) Foreign Application Priority Data
Jan. 21, 2022 (CN) 202220177706.5

(51) Int. Cl. A61L 9/20

(52) U.S. Cl. CPC A61L 9/205; A61L 2209/12; A61L 2209/15

(57) ABSTRACT

The present disclosure relates to the technical field of an air disinfection machine, and discloses a fixing structure of an ultraviolet lamp tube of an air disinfection machine, which comprises a bracket and a lamp post assembly provided on the bracket, wherein a catalytic assembly is provided on the bracket and covers the lamp post assembly, the bracket is provided with a first mounting table, the lamp post assembly penetrates through the first mounting table, the catalytic assembly comprises a protective cover which covers the lamp post assembly, the protective cover is further provided with a photocatalyst coating, a mounting seat protective cover is provided on a mounting seat, and the mounting seat is provided on the first mounting table through threaded connection.

### CROSS REFERENCE

The present disclosure is a continuation-application of International (PCT) Patent Application No. PCT/CN2023/070165, filed on Jan. 3, 2023, which claims priority of Chinese Patent Application No. 202220177706.5, filed on Jan. 21, 2022, the entire contents of which are hereby incorporated by reference in their entirety.

### TECHNICAL FIELD

The present disclosure relates to the technical field of air disinfection machine, in particular to a fixing structure of an ultraviolet lamp tube of an air disinfection machine.

### BACKGROUND

In the prior art, when an ultraviolet lamp tube of an air disinfection machine is mounted, first, a lamp holder is mounted on a bracket through a fixing member, then the lamp tube is plugged on the lamp holder to be connected with the lamp holder, and the fixing effect is realized at the same time after plugging. Thereafter, the protective cover covers the lamp tube and is connected with the bracket through screws. However, in the use process, the ultraviolet lamp tube needs to be maintained and replaced after being used for a period of time, and the inside of the protective cover needs to be cleaned. Through the current mounting method, it is necessary to remove the screws before the subsequent maintenance. Because most of the air disinfection machines are household tools, most of the families are not equipped with special screwdrivers. If the air disinfection machine is to be maintained, it is necessary to buy suitable tools or give corresponding tools in the sales process of the whole machine, which leads to increased machine cost and poor user experience.

### SUMMARY

The present disclosure provides a fixing structure of an ultraviolet lamp tube of an air disinfection machine, which solves the problems that the air disinfection machine in the related art needs to be equipped with a special screwdriver for disassembly in the maintenance process, which leads to increased cost and poor user experience are solved.

The technical scheme of the present disclosure is as follows.

A fixing structure of an ultraviolet lamp tube of an air disinfection machine is provided, comprising a bracket and a lamp post assembly provided on the bracket, wherein a catalytic assembly is provided on the bracket and covers the lamp post assembly, wherein the bracket is provided with a first mounting table, the lamp post assembly penetrates through the first mounting table, and the catalytic assembly comprises:
a protective cover which covers the lamp post assembly, wherein the protective cover is further provided with a photocatalyst coating,
a mounting seat, wherein the protective cover is provided on the mounting seat, and the mounting seat is provided on the first mounting table through threaded connection.

As a further technical scheme, the lamp post assembly is provided with a clamping table, the bracket is provided with a second mounting table located on the first mounting table, the cross-sectional area of the second mounting table is smaller than that of the first mounting table, and the fixing structure further comprises:

a lamp post fixing member, which is sleeved on the lamp post assembly and abutted on the clamping table, and is provided on the second mounting table through threaded connection.

As a further technical scheme, the fixing structure of the ultraviolet lamp tube of the air disinfection machine further comprises:

a clamping member which is provided at the top end in the protective cover and is provided with a plurality of elastic sheets, wherein the elastic sheets together encircle a clamping gap, and the lamp post assembly is clamped in the clamping gap.

As a further technical scheme, the lamp post assembly comprises:
a lamp holder, which is provided on the bracket,
a lamp tube, which is provided with a connecting end, inserted on the lamp holder and electrically communicated with the lamp holder, wherein the protective cover covers the lamp tube, and the clamping table is located on the lamp tube.

As a further technical scheme, the lamp post assembly further comprises:
a lamp holder fixing member, wherein the lamp holder is provided on the bracket through the lamp holder fixing member, and the lamp holder fixing member comprises:
a first fixing block and a second fixing block, which are oppositely provided and are both provided with a clamping end, a clamping groove, a limiting protrusion and a limiting groove, wherein the clamping ends of both of the first fixing block and the second fixing block are configured to be clamped with the opposite clamping grooves, the limiting protrusions of the first fixing block and the second fixing block are configured to extend into the opposite limiting grooves, the first fixing block and the second fixing block are clamped to encircle a clamping cavity, and the lamp holder is provided in the clamping cavity.

As a further technical scheme, the fixing structure of the ultraviolet lamp tube of the air disinfection machine further comprises:
a ballast, which is provided on the bracket and is connected with the lamp holder.

The working principle and beneficial effects of the present disclosure are as follows.

In the prior art, when an ultraviolet lamp tube of an air disinfection machine is mounted, first, a lamp holder is mounted on a bracket through a fixing member, then the lamp tube is plugged on the lamp holder to be connected with the lamp holder, and the fixing effect is realized at the same time after plugging. Thereafter, the protective cover covers the lamp tube and is connected with the bracket through screws. However, in the use process, the ultraviolet lamp tube needs to be maintained and replaced after being used for a period of time, and the inside of the protective cover needs to be cleaned. Through the current mounting method, it is necessary to remove the screws before the subsequent maintenance. Because most of the air disinfection machines are household tools, most of the families are not equipped with special screwdrivers. If the air disinfection machine is to be maintained, it is necessary to buy suitable tools or give corresponding tools in the sales process of the whole machine, which leads to increased machine cost and poor user experience.

In the present disclosure, based on the above reasons, a structure that is convenient for family users to quickly disassemble the protective cover in the use process is required, which can improve the efficiency of cleaning the interior at home and improve the user experience. A fixing structure of an ultraviolet lamp tube of an air disinfection machine is designed. Specifically, the device consists of a bracket, a lamp post assembly and a catalytic assembly. In the mounting process, first, the lamp post assembly is mounted on the bracket through screws. The catalytic assembly covers the lamp post assembly, and the photocatalyst coating on the protective cover in the catalytic assembly forms a match with the ultraviolet light radiated by the lamp post assembly to realize the catalytic function. The protective cover is provided with several uniform through holes, through which the ultraviolet light formed by the lamp post assembly are transmitted. The protective cover can also be a net cover to realize the same function. The protective cover is fixedly provided on the mounting seat. The protective cover is quickly mounted on the bracket through the threads of the mounting seat and the first mounting table. Moreover, the lamp post assembly is covered inside, which protects the lamp post assembly and realizes catalysis. In this way, there is no need to use special tools for disassembly when using the device at home, which improves the efficiency of disassembly and assembly and provides better use experience for users. Maintenance and cleaning can be completed only by disassembling the protective cover in the form of threads. This scheme not only reduces the production cost of the device, but also increases the user experience.

### BRIEF DESCRIPTION OF THE DRAWINGS

The present disclosure will be further explained in detail with the attached drawings and specific embodiments.

FIG. 1 is a schematic diagram of an explosive structure according to the present disclosure.

FIG. 2 is a schematic structural diagram of part A according to the present disclosure.

FIG. 3 is a schematic view of a cross-sectional structure according to the present disclosure.

FIG. 4 is a schematic structural diagram of part B according to the present disclosure.

FIG. 5 is a schematic diagram of a bottom structure of a bracket according to the present disclosure.

In the figures: 1, bracket, 2, lamp post assembly, 3, catalytic assembly, 4, first mounting table, 5, protective cover, 7, mounting seat, 8, clamping platform, 9, second mounting table, 10, lamp post fixing member, 11, clamping member, 12, elastic sheet, 13, clamping gap, 14, lamp holder, 15, lamp tube, 16, connecting end, 17, lamp holder fixing member, 18, first fixing block, 19, second fixing block, 20, clamping end, 21, clamping groove, 22, limiting protrusion, 23, limiting groove, 25, ballast.

### DETAILED DESCRIPTION OF THE EMBODIMENTS

The technical scheme in the embodiment of the present disclosure will be clearly and completely described hereinafter in combination with the embodiment of the present disclosure. Obviously, the described embodiments are only some of the embodiments of the present disclosure, rather than all of the embodiments. Based on the embodiments in the present disclosure, all other embodiments obtained by those skilled in the art without creative work are related to the scope of protection of the present disclosure.

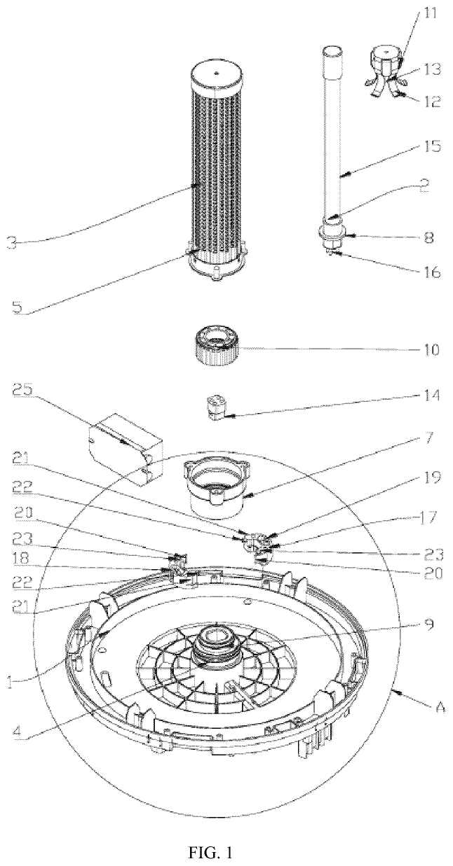

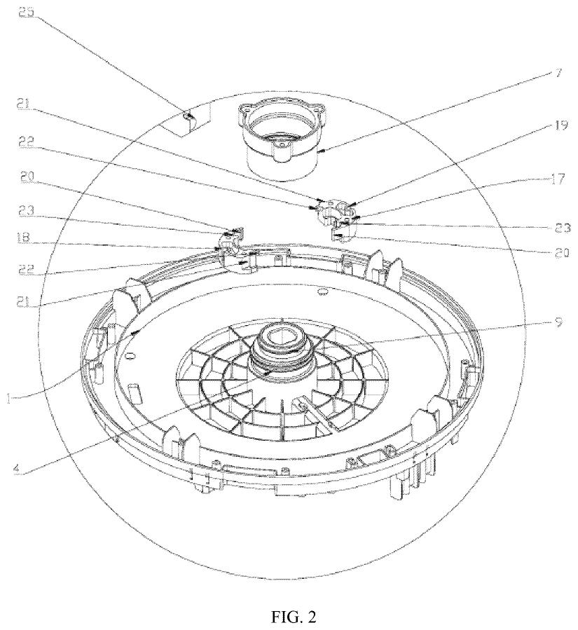

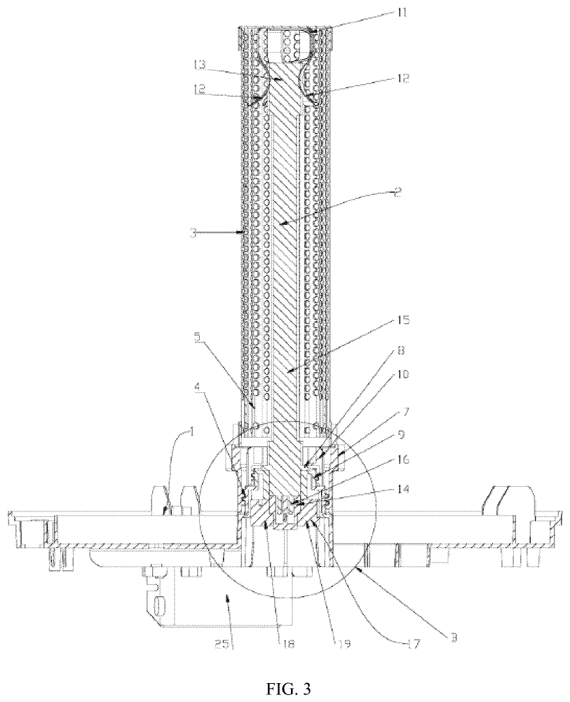

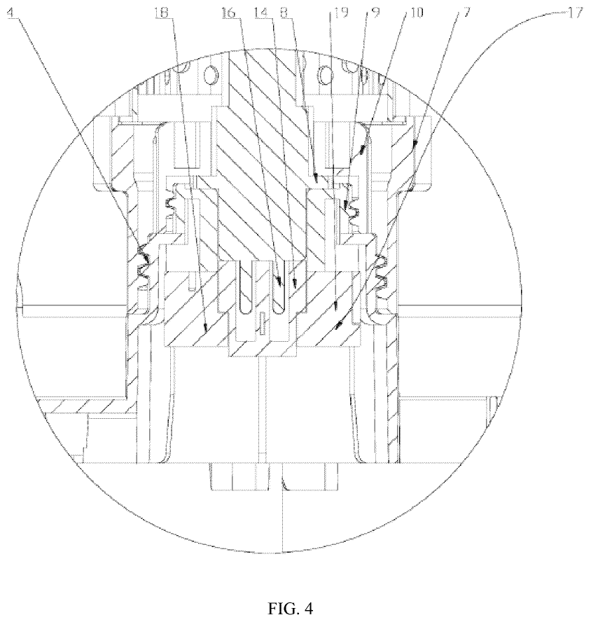

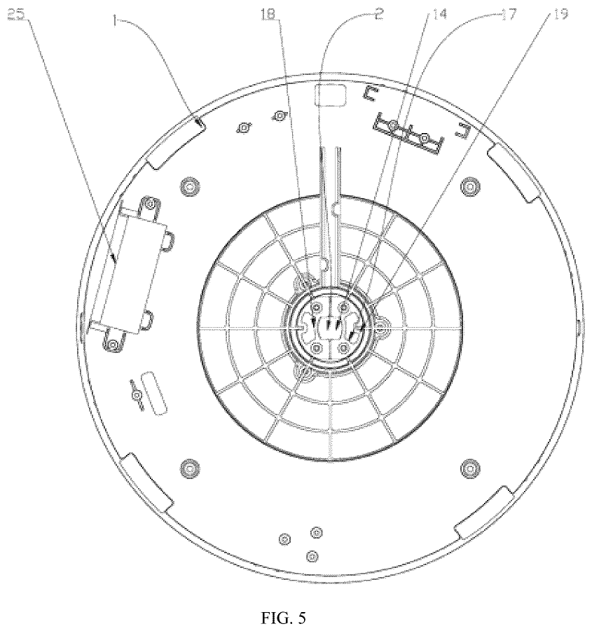

As shown in FIG. 1 to FIG. 5, this embodiment provides a fixing structure of an ultraviolet lamp tube of an air disinfection machine, comprising a bracket 1 and a lamp post assembly 2 provided on the bracket 1, wherein a catalytic assembly 3 is provided on the bracket 1 and covers the lamp post assembly 2, wherein the bracket 1 is provided with a first mounting table 4, the lamp post assembly 2 penetrates through the first mounting table 4, and the catalytic assembly 3 comprises:
a protective cover 5 which covers the lamp post assembly 2, wherein the protective cover 5 is further provided with a photocatalyst coating,
a mounting seat 7, wherein the protective cover 5 is provided on the mounting seat 7, and the mounting seat 7 is provided on the first mounting table 4 through threaded connection.

In the prior art, when an ultraviolet lamp tube 15 of an air disinfection machine is mounted, first, a lamp holder 14 is mounted on a bracket 1 through a fixing member, then the lamp tube 15 is plugged on the lamp holder 14 to be connected with the lamp holder 14, and the fixing effect is realized at the same time after plugging. Thereafter, the protective cover 5 covers the lamp tube 15 and is connected with the bracket 1 through screws. However, in the use process, the ultraviolet lamp tube 15 needs to be maintained and replaced after being used for a period of time, and the inside of the protective cover 5 needs to be cleaned. Through the current mounting method, it is necessary to remove the screws before the subsequent maintenance. Because most of the air disinfection machines are household tools, most of the families are not equipped with special screwdrivers. If the air disinfection machine is to be maintained, it is necessary to buy suitable tools or give corresponding tools in the sales process of the whole machine, which leads to increased machine cost and poor user experience.

In this embodiment, based on the above reasons, a structure that is convenient for family users to quickly disassemble the protective cover 5 in the use process is required, which can improve the efficiency of cleaning the interior at home and improve the user experience. A fixing structure of an ultraviolet lamp tube of an air disinfection machine is designed. Specifically, the device consists of a bracket 1, a lamp post assembly 2 and a catalytic assembly 3. In the mounting process, first, the lamp post assembly 2 is mounted on the bracket 1 through screws. The catalytic assembly 3 covers the lamp post assembly 2, and the photocatalyst coating on the protective cover 5 in the catalytic assembly 3 forms a match with the ultraviolet light radiated by the lamp post assembly 2 to realize the catalytic function. The protective cover 5 is provided with several uniform through holes, through which the ultraviolet light formed by the lamp post assembly 2 are transmitted. The protective cover 5 can also be a net cover to realize the same function. The protective cover 5 is fixedly provided on the mounting seat 7. The protective cover 5 is quickly mounted on the bracket 1 through the threads of the mounting seat 7 and the first mounting table 4. Moreover, the lamp post assembly 2 is covered inside, which protects the lamp post assembly 2 and realizes catalysis. In this way, there is no need to use special tools for disassembly when using the device at home, which improves the efficiency of disassembly and assembly and provides better use experience for users. Maintenance and cleaning can be completed only by disassembling the protective cover 5 in the form of threads. This scheme not only reduces the production cost of the device, but also increases the user experience.

Further, the lamp post assembly 2 is provided with a clamping table 8, the bracket 1 is provided with a second mounting table 9 located on the first mounting table 4, the cross-sectional area of the second mounting table 9 is smaller than that of the first mounting table 4, and the fixing structure further comprises:
a lamp post fixing member 10, which is sleeved on the lamp post assembly 2 and abutted on the clamping table 8, and is provided on the second mounting table 9 through threaded connection.

In this embodiment, in order to better protect the lamp post assembly 2 and reduce the detachment of the lamp tube 15 in the lamp post assembly 2 and further the damage of the lamp post assembly 2 caused by vibration during transportation, the lamp post fixing member 10 is sleeved on the lamp post assembly 2, and the lamp post fixing member 10 is abutted on the clamping table 8 of the lamp post assembly 2 to form a better connection effect with the lamp post assembly 2. The second mounting table 9 is mounted on the bracket 1 in the form of threaded connection. Then, the protective cover 5 covers the lamp post fixing member 10 to protect the inside. In this way, the lamp post can be prevented from leaving the mounting position due to vibration, thus avoiding the damage of the lamp tube 15. Through this scheme, the stability of the device can be further improved.

Further, the fixing structure of the ultraviolet lamp tube of the air disinfection machine further comprises:
a clamping member 11 which is provided at the top end in the protective cover 5 and is provided with a plurality of elastic sheets 12, wherein the elastic sheets 12 together encircle a clamping gap 13, and the lamp post assembly 2 is clamped in the clamping gap 13.

In this embodiment, in order to further improve the mounting stability of the lamp post assembly 2, a clamping member 11 is provided in the protective cover 5. The clamping member 11 is provided with a plurality of elastic sheets 12. The elastic sheets 12 are arc-shaped and can form a clamping gap 13. The top end of the lamp post assembly 2 extends into the clamping gap 13. The lamp post assembly 2 is squeezed by the elasticity of the elastic sheets 12, so that the lamp post is fixed. Compared with the fixing form in the prior art, according to the scheme, the lamp post can be quickly disassembled and fixed. When the lamp post needs to be disassembled, the clamping and detachment of the lamp post assembly 2 can be realized only by pulling out the protective cover 5, so that the use experience and the simplicity of operation of the device can be further improved, and the practicability of the device can be further improved.

Further, the lamp post assembly 2 comprises:
a lamp holder 14, which is provided on the bracket 1,
a lamp tube 15, which is provided with a connecting end 16, inserted on the lamp holder 14 and electrically communicated with the lamp holder 14, wherein the protective cover 5 covers the lamp tube 15, and the clamping table 8 is located on the lamp tube 15.

In this embodiment, a lamp post assembly 2 is detailed. The lamp tube 15 and the lamp holder 14 can be quickly connected through the connecting end 16 located on the lamp tube 15.

Further, the lamp post assembly 2 further comprises
a lamp holder 14 fixing member, wherein the lamp holder 14 is provided on the bracket 1 through the lamp holder 14 fixing member, and the lamp holder 14 fixing member comprises:
a first fixing block 18 and a second fixing block 19, which are oppositely provided and are both provided with a clamping end 20, a clamping groove 21, a limiting protrusion 22 and a limiting groove 23, wherein the clamping ends 20 of both of the first fixing block and the second fixing block are configured to be clamped with the opposite clamping grooves 21, the limiting protrusions 22 of the first fixing block and the second fixing block are configured to extend into the opposite limiting grooves 23, the first fixing block 18 and the second fixing block 19 are clamped to encircle a clamping cavity, and the lamp holder 14 is provided in the clamping cavity.

In this embodiment, a lamp holder 14 fixing member is detailed. Most of the lamp holder 14 fixing members in the prior art need to insert the lamp holder 14 into a clamping groove to fix the lamp holder 14. Because the clamping groove is not adjustable, it is difficult to install, which is time-consuming and laborious. Now, a lamp holder 14 fixing member that can be quickly assembled and disassembled is designed. Specifically, the first fixing block 18 and the second fixing block 19 match with each other quickly through the clamping ends 20, the clamping grooves 21, the limiting protrusions 22 and the limiting grooves 23 of the two fixing blocks. After the lamp holder 14 is placed in the clamping cavity encircled by the two fixing blocks, the clamping ends 20 of the two fixing blocks are clamped with the opposite clamping grooves 21, and the limiting protrusions 22 of the two fixing blocks extend into the opposite limiting grooves 23 to match with other. Further, rapid clamping and fixing can be realized. Disassembly can be completed by only canceling the clamping of the clamping end 20. In this way, the rapid disassembly and assembly of the device can be realized. At the same time, the clamping effect can be improved through this form of clamping, further improving the stability and operation convenience of the device.

Further, the fixing structure of the ultraviolet lamp tube of the air disinfection machine further comprises a ballast 25, which is provided on the bracket 1 and is connected with the lamp holder 14.

The above is only the preferred embodiment of the present disclosure, rather than limit the present disclosure. Any modification, equivalent substitution, improvement, etc. made within the spirit and principle of the present disclosure should be included in the scope of protection of the present disclosure.

What is claimed is:

1. A fixing structure of an ultraviolet lamp tube of an air disinfection machine, comprising a bracket (1) and a lamp post assembly (2) provided on the bracket (1), wherein a catalytic assembly (3) is provided on the bracket (1) and covers the lamp post assembly (2), wherein the bracket (1) is provided with a first mounting table (4), the lamp post assembly (2) penetrates through the first mounting table (4), and the catalytic assembly (3) comprises:
a protective cover (5) which covers the lamp post assembly (2), wherein the protective cover (5) is further provided with a photocatalyst coating,
a mounting seat (7), wherein the protective cover (5) is provided on the mounting seat (7), and the mounting seat (7) is provided on the first mounting table (4) through threaded connection.

2. The fixing structure of the ultraviolet lamp tube of the air disinfection machine according to claim 1, wherein the lamp post assembly (2) is provided with a clamping table (8), the bracket (1) is provided with a second mounting table (9) located on the first mounting table (4), the cross-sectional area of the second mounting table (9) is smaller than that of the first mounting table (4), and the fixing structure further comprises:
a lamp post fixing member (10), which is sleeved on the lamp post assembly (2) and abutted on the clamping table (8), and is provided on the second mounting table (9) through threaded connection.

3. The fixing structure of the ultraviolet lamp tube of the air disinfection machine according to claim 1, further comprising a clamping member (11) which is provided at the top end in the protective cover (5) and is provided with a plurality of elastic sheets (12), wherein the elastic sheets (12) together encircle a clamping gap (13), and the lamp post assembly (2) is clamped in the clamping gap (13).

4. The fixing structure of the ultraviolet lamp tube of the air disinfection machine according to claim 2, wherein the lamp post assembly (2) comprises:
a lamp holder (14), which is provided on the bracket (1),
a lamp tube (15), which is provided with a connecting end (16), inserted on the lamp holder (14) and electrically communicated with the lamp holder (14), wherein the protective cover (5) covers the lamp tube (15), and the clamping table (8) is located on the lamp tube (15).

5. The fixing structure of the ultraviolet lamp tube of the air disinfection machine according to claim 4, wherein the lamp post assembly (2) further comprises:
a lamp holder fixing member (17), wherein the lamp holder (14) is provided on the bracket (1) through the lamp holder fixing member (17), and the lamp holder fixing member (17) comprises:
a first fixing block (18) and a second fixing block (19), which are oppositely provided and are both provided with a clamping end (20), a clamping groove (21), a limiting protrusion (22) and a limiting groove (23), wherein the clamping ends (20) of both of the first fixing block and the second fixing block are configured to be clamped with the opposite clamping grooves (21), the limiting protrusions (22) of the first fixing block and the second fixing block are configured to extend into the opposite limiting grooves (23), the first fixing block (18) and the second fixing block (19) are clamped to encircle a clamping cavity, and the lamp holder (14) is provided in the clamping cavity.

6. The fixing structure of the ultraviolet lamp tube of the air disinfection machine according to claim 4, further comprising a ballast (25), which is provided on the bracket (1) and is connected with the lamp holder (14).
